(19) United States
(12) Patent Application Publication
(10) Pub. No.: US 2023/0022794 A1
(43) Pub. Date: Jan. 26, 2023

(54) METHOD AND MODULE FOR MONITORING TRACKS

(71) Applicant: DeCloak Intelligences Co., Taipei City (TW)

(72) Inventors: Yao-Tung Tsou, Taipei City (TW); Juang-Ying Chueh, Taipei City (TW); Jen-Yu Huang, Taipei City (TW)

(21) Appl. No.: 17/829,685

(22) Filed: Jun. 1, 2022

Related U.S. Application Data
(60) Provisional application No. 63/332,283, filed on Apr. 19, 2022.
(60) Provisional application No. 63/196,678, filed on Jun. 3, 2021.

(51) Int. Cl. G16H 50/80

(52) U.S. Cl. CPC G16H 50/80

(57) ABSTRACT

The present invention provides a method for monitoring tracks and a track monitoring module. The method comprises: maintaining a target list, stored a target track, by a monitoring server; recording a first track related to a first mobile device by a first monitoring application; and comparing the similarity between the first track and the target track by the first monitoring application to generate a comparison result.

### CROSS REFERENCE TO RELATED APPLICATION

The present application claims priority to U.S. provisional application Ser. No. 63/196,678 filed on Jun. 3, 2021, and U.S. provisional application Ser. No. 63/332,283 filed on Apr. 19, 2022 the entire content of which is incorporated by reference to this application.

### BACKGROUND OF THE INVENTION

### 1. Field of the Invention

The present invention pertains to a method and a module for monitoring tracks, more specifically to a method and a module for monitoring tracks which can compare a target track locally.

### 2. Description of the Prior Art

As the highly contagious epidemic heats up, administrative units may invest a lot of manpower and resources to investigate the footprints of the infected people. However, the number of the infected people has increased sharply and the infected people may cover a considerable commuting range. Traditional investigation methods have been unable to keep up with the spread of the epidemic, nor can they effectively warn the public in advance to slow down the epidemic. For example, when the number of the footprints of the infected people is small, the administrative units can trace the contacts of the infected people and disinfect the relevant places after a full investigation. However, when the number of the footprints of the infected people is huge, it will be very difficult for the administrative units to trace the infected people and their contacts. And, because the footprints of the infected in each geographic area are not fully published, people are also unable to assess the risk of infection while traveling by themselves.

Therefore, there is currently a need for a new track monitoring method and module for tracking relevant footprints and compiling information about relevant footprints to the public, so that the public can understand the risk of infection in a specific area. At the same time, relying on the public to raise their awareness against the epidemic, it is possible to stop the spread of the epidemic.

### SUMMARY OF THE INVENTION

The present invention provides a method for monitoring tracks, which can collect the known tracks of a target by a monitoring server, and then the monitoring server provides the known tracks to a user end to compare its own tracks to determine whether the user end conforms to the target.

The present invention provides a method for monitoring tracks comprising: maintaining a target list, stored a target track, by a monitoring server; recording a first track related to a first mobile device by a first monitoring application; and comparing the similarity between the first track and the target track by the first monitoring application to generate a comparison result.

In some embodiments, the first mobile device may be associated with a first Bluetooth code, and when the comparison result may indicate that the first track matches the target track, the first monitoring application uploads the first Bluetooth code to the monitoring server. Besides, the method may further comprise the following steps: pushing, by the monitoring server, the first Bluetooth code to a second monitoring application associated with a second mobile device; and determining, by the second monitoring application, whether the first Bluetooth code is recorded in a Bluetooth receiving list of the second mobile device.

In some embodiments, the method may further comprise: generating a reference track by the first monitoring application according to the first track when the comparison result indicates that the first track matches the target track; and uploading the reference track, being de-identified, to the monitoring server by the first monitoring application. In addition, the first track may include at least a real geographic address, the reference track may include at least a reference geographic address. And, in the step of generating the reference track by the first monitoring application according to the first track, may further comprise: selecting a landmark address spaced apart by the real geographic addresses less than a first distance; and setting the landmark address as the reference geographic address.

In some embodiments, in the step of uploading the reference track, being de-identified, to the monitoring server by the first monitoring application, may further comprise: randomizing the reference geographic address to generate a plurality of second geographic addresses, each of the second geographic addresses corresponded to a weight value; and uploading the second geographic addresses and the weight values corresponded to the second geographic addresses.

In some embodiments, the method may further comprise: generating, by the monitoring server, a population distribution map according to the second geographic addresses and the weight values corresponded to the second geographic addresses; and obtaining the population distribution map by a third monitoring application associated with a third mobile device; wherein the population distribution map may record a track quantity associated with each of the landmark addresses in the target list. Besides, the method may also comprise: displaying, by the third monitoring application, the population distribution map within a second distance around a positioning address of the third mobile device. Moreover, the method may also comprise: displaying, by the third monitoring application, the population distribution map of an administrative area where a positioning address of the third mobile device is located.

In some embodiments, the first track may include at least a real geographic address, the target track may include at least a target geographic address, and in the step of comparing the similarity between the first track and the target track by the first monitoring application to generate the comparison result, may further comprise: determining whether the distance between the real geographic address and the target geographic address is less than a third distance; and generating the comparison result indicating that the first track matches the target track when the distance between the real geographic address and the target geographic address is less than the third distance.

The present invention provides a track monitoring module, which operates on the user end, so that the user end can compare its own tracks with a specific target track by itself, so as to determine whether the user end conforms to the target.

The present invention provides a track monitoring module comprising a transmission unit and a processor. The transmission unit receives a target track. The processor executes a first monitoring application. Wherein the first monitoring application records a first track and compares the similarity between the first track and the target track to generate a comparison result.

In some embodiments, the track monitoring module may be disposed in a mobile device, and the mobile device is associated with a first Bluetooth code. And, the first Bluetooth code may be uploaded by the first monitoring application when the comparison result indicates that the first track matches the target track. Besides, the transmission unit may further receive a target Bluetooth code, and the first monitoring application may determine whether the target Bluetooth code is recorded in a Bluetooth receiving list of the mobile device.

In some embodiments, a reference track may be generated by the first monitoring application according to the first track when the comparison result indicates that the first track matches the target track, and the first monitoring application may upload the reference track which is de-identified. Besides, the first track may include at least a real geographic address, the reference track may include at least a reference geographic address, the first monitoring application may select a landmark address spaced apart by the real geographic addresses less than a first distance, and set the landmark address as the reference geographic address. Moreover, the first monitoring application may randomize at least one of the reference geographic addresses of the reference track to generate a plurality of second geographic addresses, each of the second geographic addresses is corresponded to a weight value, and the second geographic addresses and the weight values corresponded to the second geographic addresses may be uploaded by the transmission unit to de-identify the reference track.

In some embodiments, the first monitoring application may receive a population distribution map through the transmission unit, and the population distribution map may record a track quantity associated with each of the landmark addresses in the target list. Besides, the first monitoring application may display the population distribution map within a second distance around a positioning address of the mobile device, the first monitoring application may alternatively display the population distribution map of an administrative area where a positioning address of the mobile device is located.

In some embodiments, the first track may include at least a real geographic address, the target track may include at least a target geographic address, the first monitoring application may determine whether the distance between the real geographic address and the target geographic address is less than a third distance, and may generate the comparison result indicating that the first track matches the target track when the distance between the real geographic address and the target geographic address is less than the third distance.

To sum up, the method of track monitoring method and the track monitoring module provided by the present invention can be used to collect the known target tracks by the monitoring server, use the mobile device of the user end to compare whether its own track conforms to the target track, and then determine whether the mobile device of the user end is the target. In addition, the method of track monitoring method and the track monitoring module provided by the present invention can also provide a population distribution map around the mobile device of the user, thereby helping the user to evaluate the risk in a specific area.

### BRIEF DESCRIPTION OF THE APPENDED DRAWINGS

FIG. 1 is block diagram of a system applying a method for monitoring tracks according to an embodiment of the present invention.

FIG. 2 is a schematic diagram illustrating a scenario of the method for monitoring tracks according to an embodiment of the present invention.

FIG. 3 is a flowchart showing the method for monitoring tracks according to an embodiment of the present invention.

FIG. 4 is a flowchart showing the method for monitoring tracks according to another embodiment of the present invention.

### DETAILED DESCRIPTION OF THE INVENTION

The features, targetions, and functions of the present invention are further disclosed below. However, it is only a few of the possible embodiments of the present invention, and the scope of the present invention is not limited thereto; that is, the equivalent changes and modifications done in accordance with the claims of the present invention will remain the subject of the present invention. Without departing from the spirit and scope of the invention, it should be considered as further enablement of the invention.

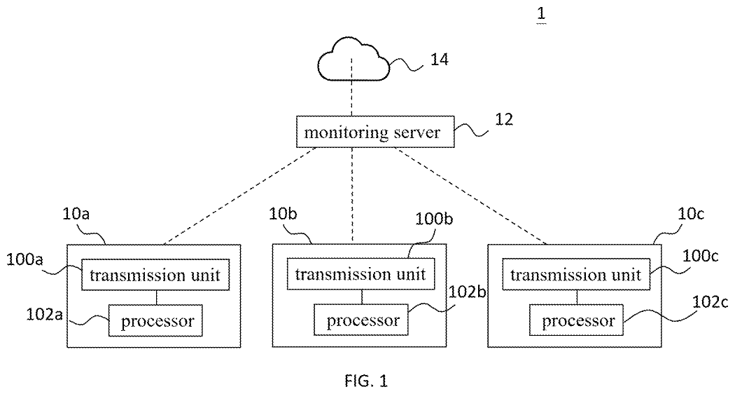

Please refer to FIG. 1, FIG. 1 is block diagram of a system applying a method for monitoring tracks according to an embodiment of the present invention. As shown in FIG. 1, the method for monitoring tracks of the present invention can be applied to the system 1, and the system 1 can have multiple track monitoring modules (a track monitoring module 10a, a track monitoring module 10b, track monitoring module 10b, and a track monitoring module group 10c), a monitoring server 12, and a cloud network 14. The track monitoring module 10a, the track monitoring module 10b, and the track monitoring module 10c can be respectively wirelessly connected to the monitoring server 12, and then the monitoring server 12 can be coupled to the cloud network 14. Taking the track monitoring module 10a as an example, the track monitoring module 10a may have a transmission unit 100a and a processor 102a. The transmission unit 100a may be used to transmit various data, and the processor 102a may perform logical functions. Of course, the track monitoring module 10a may also include other elements, such as a screen or a user interface, which is not limited in this embodiment. In one example, the track monitoring module 10a can be regarded as a mobile device, such as a mobile device or a part of the mobile device. In addition, this embodiment does not limit the means by which the track monitoring module 10a is connected to the monitoring server 12. For example, when the track monitoring module 10a is the mobile device, the track monitoring module 10a can be wirelessly connected to the monitoring server 12 through 4G, 5G, or Wi-Fi technologies.

The transmission unit 100a of the track monitoring module 10a can receive the target track, and the target track is updated by the monitoring server 12 periodically or in real time. For example, the monitoring server 12 can maintain a target list in real time, and the target list can record (or store) at least one target track. Although this embodiment does not limit when or where to apply the method of the application, for the convenience of description, the following assumes that the footprints of the infected people during the epidemic are the target tracks of this embodiment. In one example, the monitoring server 12 can obtain the relevant data of the target track from the cloud network 14, and the type of the cloud network 14 is also not limited in this embodiment. For example, the cloud network 14 may be a database maintained by a central or local administrative unit, in which the footprints of at least a portion of the infected peoples are disclosed. For another example, the cloud network 14 may be a website of a news media, and the website records the footprints of at least a part of the infected people. Person having ordinary skill in the art can understand that as long as the monitoring server 12 can obtain the target track from the network, any source which provides the target tracks should belong to the scope of the cloud network 14 in this embodiment.

The processor 102a can execute a monitoring application (a first monitoring application), and the monitoring application can record the first track associated with the track monitoring module 10a. In practice, the processor 102a can drive a positioning element (not shown), such as a GPS element, to obtain the tracks recorded by the positioning element. In one example, assuming that the track monitoring module 10a is installed in the mobile device, the monitoring application can record a real geographic address of the mobile device, and the recorded time and the real geographic address can be is regarded as the first track. The first track of the example track monitoring module 10a in this embodiment can be represented as Table 1.

TABLE 1

| time | real geographic address |
|---|---|
| 2022/6/1,13:55 | (X0,Y0) |
| 2022/6/1,14:00 | (X1,Y1) |
| 2022/6/1,14:05 | (X2,Y2) |
| 2022/6/1,14:10 | (X3,Y3) |
| 2022/6/1,14:15 | (X4,Y4) |
| 2022/6/1,14:20 | (X5,Y5) |

The real geographic addresses recorded in Table 1 may be latitude and longitude coordinates or other coordinates available for positioning, which is not limited in this embodiment. It is worth mentioning that, although the example in Table 1 is that the real geographic addresses are recorded every 5 minutes, this embodiment does not limit the recording frequency of the real geographic addresses. In addition, this embodiment does not limit the time span recorded by the first track. For example, Table 1 may only show a part of the first track, and the first track may be continuously recorded by the track monitoring module 10a within one day or one week. In one example, the monitoring application can shorten the time interval if the real geographic address changes significantly over a period of time, such as when the user is moving with the mobile device. Conversely, if there is little change in the real geographic address for a period of time, such as after the user returns home at night, the monitoring application can extend the time interval.

In addition, the monitoring application may periodically or manually download the target tracks in a target list from the monitoring server 12 via the transmission unit 100a. Similar to the representation of the first track in the aforementioned Table 1, the target track may also correspond to time (when) and location (where). However, the target tracks are usually obtained by administrative units or news media after investigations, and person having ordinary skill in the art should understand that the target tracks might be described roughly. For example, the time may not be specific and might be described as a time interval, and target geographic addresses may also be ambiguous. In practice, the monitoring server 12 may preprocess the target track obtained from the cloud network 14 in the first place. For example, the location may be recorded as a specific store or a specific landmark in the original target track, and the monitoring server 12 can automatically search for the geographic address of the specific store or the specific landmark and record the target geographic address in coordinates. In one example, a part of the target track can be represented in Table 2 shown below.

TABLE 2

| time | target geographic address |
|---|---|
| 2022/6/1,2:00-14:00 | restaurant A (Xa,Ya) |
| 2022/6/1,14:10 | intersection B (Xb,Yb) |
| 2022/6/1,14:20-15:00 | store C (Xc,Yc) |

In Table 2, the demonstrated target track has three events. For example, the target track indicates that the target is in a restaurant A from 12:00 noon to 2:00 p.m. on Jun. 1, 2022, and after leaving the restaurant A in the afternoon the target appears at an intersection B at 2:10 μm, and then appears at a store C between 2:20 μm and 3 pm. The monitoring application will compare the first track in Table 1 with the target track in Table 2, and check whether the first track overlaps the target track. In practice, since the target track is not that precise, the monitoring application may compare whether the real geographic address in a similar period of time is close to the target geographic address. For example, the monitoring application can compare the real geographic address (X0, Y0) at 1:55 pm, the real geographic address (X1, Y1) at 2 μm, and the real geographic address (X2, Y2) at 2:05 pm with the target geographic address (Xa, Ya) of the restaurant A. Similarly, the monitoring application can compare the real geographic address (X2, Y2) at 2:05 pm, the real geographic address (X3, Y3) at 2:10 μm, and the real geographic address (X4, Y4) at 2:15 pm with the target geographic address (Xb, Yb) of the intersection B. In addition, the monitoring application can also repeat the above comparison until all of target geographic addresses are checked, which will not be shown in this embodiment.

Assuming that the monitoring application finds the distance between the real geographic address (X0, Y0) and the target geographic address (Xa, Ya) is less than a preset distance (third distance) during the above comparison, the monitoring application can determine that the real geographic address (X0, Y0) at 1:55 pm matches the first event of the target track. Similarly, if the distance between the subsequent real geographic address (X3, Y3) and the target geographic address (Xb, Yb) is less than the preset distance, and the distance between the real geographic address (X5, Y5) and the target geographic address (Xc, Yc) is also less than the preset distance, the monitoring application can generate a comparison result to indicate that the first track matches the target track since all events in the target track are happened close to the corresponding real geographic address at a similar time. Of course, the present embodiment does not limit the value of the preset distance, which can be set by person having ordinary skill in the art. In one example, the monitoring server 12 can receive several comparison results returned by the monitoring applications in different mobile devices. If the monitoring server 12 finds that there are too many comparison results indicate “matched” for the same target track, the monitoring server 12 can also instruct the monitoring applications to use a smaller preset distance, or can use the real geographic address corresponding to a smaller time interval for comparison to find out which one is the closet track.

Next, if the monitoring application of the track monitoring module 10a determines that its track (the first track) conforms to the target track, the monitoring application of the track monitoring module 10a will notify the monitoring server 12. In practice, in addition to sending the comparison result to the monitoring server 12, the monitoring application can also actively send the Bluetooth code (first Bluetooth code) corresponded to the track monitoring module 10a to the monitoring server 12. In one example, the first Bluetooth code can be de-identified or digitized through various encoding methods, so that only the monitoring server 12 can find out the information related to the first Bluetooth code. And then, the monitoring server 12 can push the information corresponded to the first Bluetooth code to the monitoring applications in all track monitoring modules (mobile devices). For example, both the track monitoring module 10b and the track monitoring module 10c can receive information about the first Bluetooth code from the monitoring server 12. At this time, the track monitoring module 10b can compare whether its Bluetooth receiving list has a record related to the first Bluetooth code.

In one example, the monitoring application can turn on a Bluetooth module in the mobile device, so that the Bluetooth module in the mobile device can receive and record the surrounding Bluetooth connection requests. Then, when the monitoring application obtains the information about the specific Bluetooth code (aforementioned first Bluetooth code), it will start to find out whether the first Bluetooth code has sent its request and been recorded in the Bluetooth receiving list. Taking epidemic prevention as an example, since the first track in the track monitoring module 10a matches the target track, the user holding the track monitoring module 10a is likely to be the infected people. And, because the transmission range of the Bluetooth module is limited, if the monitoring application in the track monitoring module 10b determines that the Bluetooth receiving list has recorded the first Bluetooth code, it is very likely that the user of the track monitoring module 10b has been in close contact with the user (infected people) of the track monitoring module 10a. At this time, the monitoring application in the track monitoring module 10b can pop up a warning message to remind the user of the track monitoring module 10b to pay more attention to his/her own health.

On the other hand, the monitoring application can also upload the de-identified first track to the monitoring server 12. The reason is that since the first track can be regarded as the track of the infected people, in order to maintain the privacy of the infected people and protect the confidentiality of personal information, the monitoring application needs to avoid that the first track can clearly point to a certain user so that the monitoring server 12 cannot trace back to the infected people. Taking the example in Table 1 above, although the original first track has very clear coordinates, the monitoring application will de-identify the first track into a reference track. Similarly, the reference track also has a reference geographic address corresponded to the specific time, as shown in Table 3 below.

TABLE 3

| | real geographic | reference geographic |
|---|---|---|
| time | address | address |
| 2022/6/1,13:55 | (X0,Y0) | restaurant A (Xa,Ya) |
| 2022/6/1,14:00 | (X1,Y1) | store D (Xd,Yd) |
| 2022/6/1,14:05 | (X2,Y2) | store E (Xe,Ye) |
| 2022/6/1,14:10 | (X3,Y3) | intersection B (Xb,Yb) |
| 2022/6/1,14:15 | (X4,Y4) | intersection F (Xf,Yf) |
| 2022/6/1,14:20 | (X5,Y5) | store c (Xc,Yc) |

In the example of Table 3, the real geographic address (X0, Y0) at 1:55 pm, and the striking landmark closest to the real geographic address (X0, Y0) may be restaurant A, then the monitoring application will take the landmark address (Xa, Ya) of restaurant A as the reference geographic address at this time. Then, the real geographic address (X1, Y1) at 2 pm, while the infected people may be moving, and the striking landmark closest to the real geographic address (X1, Y1) may be the store D passing by, the monitoring application will use the landmark address (Xd, Yd) of store D as the reference geographic address at this time. In this way, the monitoring application can produce a series of reference geographic addresses associated with nearby landmarks. Person having ordinary skill in the art can understand that as long as the real geographic address and the reference geographic address at the same time point are close enough, for example, less than the preset distance (the first distance), the approximate movement of the infected people can be described, and still can have the privacy of the infected people. Next, after the monitoring application de-identifies the first track into a reference track, it will be uploaded to the monitoring server 12, so that the monitoring server 12 can update the maintained target list to add the reference track as a new target track. Alternatively, since the reference track is more detailed and precise, the monitoring server 12 may also use the reference track to overwrite or replace the old target track.

It is worth mentioning that this embodiment does not limit the step of de-identifying the first track to be performed by the monitoring application. For example, if the local processor (such as the processor 102a of the track monitoring module 10a) does not have sufficient computing power, the monitoring application will also be able to ensure that the monitoring server 12 will not leak user privacy. For example, the original first track can be provided to the monitoring server 12 firs, the monitoring server 12 de-identifies the first track to generate the reference track, so as to ensure that the reference track is used in subsequent analysis and operations. That is to say, the monitoring server 12 may replace the mobile device with insufficient computing power to perform the de-identification operation, so as to avoid the problem that the processing speed of the local terminal may be delayed when the amount of data is huge.

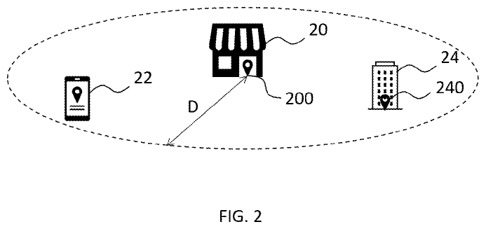

In order to explain the foregoing embodiments more clearly, please refer to FIG. 1 and FIG. 2 together. FIG. 2 is a schematic diagram illustrating a scenario of the method for monitoring tracks according to an embodiment of the present invention. As described in the foregoing embodiments, the monitoring application can de-identify the original track, so that the real geographic address can be simulated at the reference geographic address. Assuming that one of the landmarks in the neighborhood is the store 20, in order to de-identify the real geographic address of the mobile device 22 at a certain point in time, the monitoring application will simulate that the mobile device 22 can be at the landmark location 200 of the store 20. Therefore, after de-identification, the real geographic address of the mobile device 22 within the distance D from the landmark address 200 will be displayed at the landmark address 200. That is, the landmark address 200 is the reference geographic address for the mobile devices around the store 20. In addition to recording the number of reference geographic addresses in a reference track, the monitoring server 12 can also count the number of tracks associated with a certain reference geographic address.

In other words, after the monitoring server 12 obtains the number of tracks (track quantity) corresponded to each reference geographic address in the target list, it can generate a population distribution map correspondingly. Taking an actual example, assuming that there are 10 reference tracks all recorded the reference geographic address corresponded to the landmark address 200, the track quantity at the landmark address 200 is 10. For another example, if the landmark address 240 of a certain building 24 appears in 60 reference tracks, the track quantity of the landmark address 240 is 60. In this way, the monitoring server 12 can mark each landmark address with the corresponded track quantity. Since each track in the target list corresponds to the footprint of an infected people, the infection risk of a certain landmark location can be seen from the population distribution map created by the monitoring server 12. Person having ordinary skill in the art should understand that if the track quantity of a certain landmark is high, it should mean that there are more footprints of infected people around the landmark, and the risk of infection will also be higher. That is to say, if the track quantity corresponded to the landmark address 200 is only 10, and the track quantity corresponded to the landmark address 240 is 60, it can be inferred that the building 24 should have a higher risk of infection than the store 20.

In addition, the monitoring application may download the population distribution map from the monitoring server 12, or the monitoring server 12 may push the population distribution map to the monitoring application in each mobile device. In practice, the monitoring application can allow users to view the population distribution map. For example, the monitoring application can display the population distribution map within a certain distance (second distance) around the positioning address (such as its current location). For another example, the monitoring application may display a population distribution map that shows the administrative area where the positioning address is located. That is to say, the present embodiment does not limit the display of the population distribution map, and the user may select an interesting area of the population distribution map.

It is worth mentioning that the monitoring server 12 can also hand over each track in the target list to be re-analyzed by artificial intelligence, and the artificial intelligence can be installed in the cloud network 14. For example, the cloud network 14 can be a cloud server of Amazon, so that the cloud network 14 can analyze and predict each track, thereby achieving the purpose of monitoring tracks more effectively. Wherein, the cloud network 14 can generate analyze and prediction results based on the given tracks, and can also send it back to the monitoring server 12, so that the monitoring server 12 can further optimize the population distribution map. In one example, the population distribution map without the analysis and prediction may be produced by the monitoring server 12 based on the accumulated tracks in the past. After adding the analysis and prediction results of the cloud network 14, the monitoring server 12 may be able to simulate the population distribution map in the future, which is not limited in this embodiment.

Different from the aforementioned embodiments, the monitoring application may not directly upload the reference track, but further de-identifies the reference track, and then uploads the de-identified reference track to the monitoring server 12. For example, after the monitoring application generates the reference geographic addresses in Table 3, each reference geographic address can be scrambled into a series of garbled characters. After a specific decoding process, the garbled code can be used to indicate multiple second geographic addresses, and each second geographic address corresponds to a weight value, as shown in Table 4 below.

TABLE 4

| | reference | second geographic | weight |
|---|---|---|---|
| time | geographic address | address | value |
| 2022/ | restaurant A | (Xa0,Ya0) | 0.4 |
| 6/1,13:55 | (Xa,Ya) | (Xa1,Ya1) | 0.2 |
| | | (Xa2,Ya2) | 0.1 |
| | | (Xa3,Ya3) | 0.1 |
| | | (Xa4,Ya4) | 0.1 |
| | | (Xa5,Ya5) | 0.05 |
| | | (Xa6,Ya6) | 0.05 |

Table 4 shows a way to further de-identify the first track in Table 3. For the de-identification of the 1st reference geographic address in Table 3, the landmark address (Xa, Ya) of restaurant A will be broken up into multiple second geographic addresses, such as (Xa0, Ya0) to (Xa6, Ya6). Compared with the first row in Table 3, which can indicate that there is 1 footprint at the landmark address (Xa, Ya) of restaurant A at 1:55 pm on Jun. 1, 2022, Table 4 uses the weight value to disassemble the footprint to avoid being easily traced. For example, the first row of Table 4 indicates that there are 0.4 footprint at the second geographic address (Xa0, Ya0) at 1:55 pm on Jun. 1, 2022, and the second row of Table 4 indicates that there are 0.2 footprint at the second geographic address (Xa1, Ya1) at 1:55 pm on Jun. 1, 2022, the third row of Table 4 indicates that there are 0.1 footprint at the second geographic address (Xa2, Ya2) at 1:55 pm on Jun. 1, 2022, etc. will not be repeated here.

It should be noted that these second geographic addresses may include the landmark address (Xa, Ya) of the restaurant A, or may not have the landmark address (Xa, Ya) of the restaurant A at all. In addition, the weight value corresponded to each second geographic address is not necessarily related to the distance of the reference geographic address, and each second geographic address may be a landmark address of other landmarks (shops, buildings, intersections, etc.). In practice, any second geographic address is not necessarily close to the landmark address (Xa, Ya) of restaurant A, and may even be in different administrative areas. That is to say, the monitoring server 12 cannot directly find out the real geographic address of the infected people from the reference geographic addresses (multiple second geographic addresses) that have been scrambled and scattered. In other words, it is meaningless for the monitoring server 12 to only look at one of the second geographic addresses. However, when the number of second geographic addresses received by the monitoring server 12 is large, the monitoring server 12 can see how many footprints the second geographic addresses have through the accumulated value of the weight value of each second geographic address.

Taking a practical example, it is assumed that the data of 15 infected peoples originally contained a landmark address (Xa, Ya) with the reference geographic address in restaurant A. After the above randomization operation, the landmark address (Xa, Ya) of restaurant A may be scattered first to correspond to 100 infected people. Then, after accumulating the weight values of the 100 infected people corresponded to the landmark address (Xa, Ya) of restaurant A, the accumulated value of the weight values will still return to a value of 15 or close to 15. Person having ordinary skill in the art can understand that the accumulated value of the weight values can still be regarded as the aforementioned track quantity, but the track quantity has been de-identified and processed without personal information. In this way, the monitoring server 12 can generate statistical significance from a large number of second geographic addresses and weight values without being able to dig the privacy of the infected people.

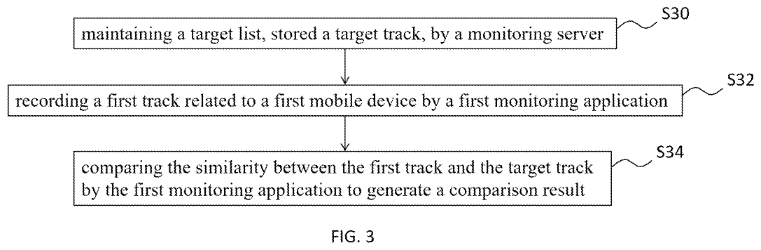

In order to explain the method for monitoring tracks of the present invention, please refer to FIG. 1 and FIG. 3 together. FIG. 3 is a flowchart showing the method for monitoring tracks according to an embodiment of the present invention. As shown in the figures, in step S30, the monitoring server 12 maintains the target list, and the target list stores at least one target track. In step S32, the first track related to the first mobile device (track monitoring module 10a) is recorded by the first monitoring application. In step S34, the first monitoring application (such as executed by the track monitoring module 10a) compares the similarity between the first track and the target track to generate a comparison result. Although other steps of the method for monitoring tracks of the present invention have been clearly described in the above embodiments, in order to better understand the method for monitoring tracks of the present embodiment, an example will further be used below to describe how it works.

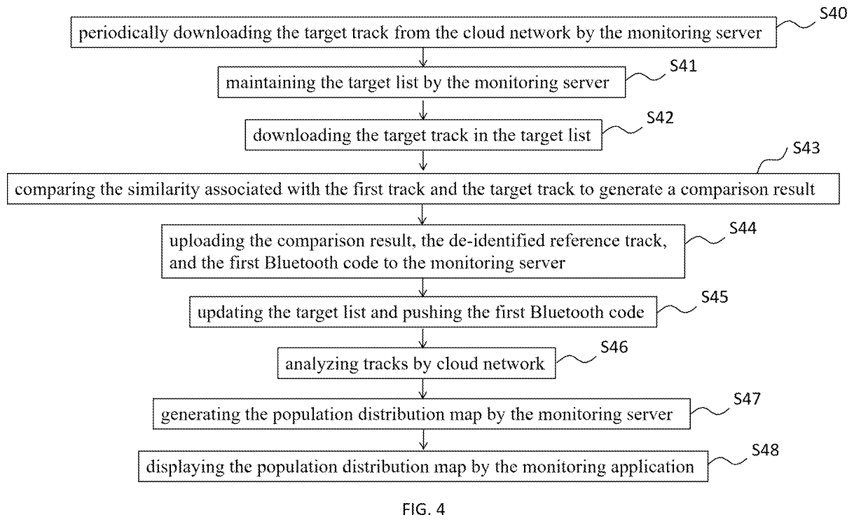

Please refer to FIG. 1 to FIG. 4 together. FIG. 4 is a flowchart showing the method for monitoring tracks according to another embodiment of the present invention. As shown in the figures, in step S40, the monitoring server 12 periodically downloads the target track from the cloud network 14. In step S41, the monitoring server 12 maintains the target list according to the downloaded target track to ensure the correctness of the target list. In step S42, the monitoring application (such as the monitoring application executed by the processor 102a) downloads the target track in the target list. In step S43, the monitoring application compares the similarity associated with the first track and the target track to generate a comparison result. In step S44, the monitoring application uploads the comparison result, the de-identified reference track, and the aforementioned first Bluetooth code to the monitoring server 12. In step S45, when the comparison result indicates that the first track matches the target track, the monitoring server 12 will record the first Bluetooth code, for example, update it in the list associated with the Bluetooth code, and the information of the first Bluetooth code name will be pushed to all available mobile devices. Afterwards, the monitoring application receiving the first Bluetooth code can compare whether it has ever been in contact with the first Bluetooth code. In step S46, the monitoring server 12 may further upload the de-identified reference tracks to the cloud network 14 capable of artificial intelligence analysis, so as to analyze and predict the results of each track. In step S47, the monitoring server 12 can generate the population distribution map based on the target track and the analysis and prediction results. In step S48, the population distribution map can be downloaded by the monitoring application, so that the user can view the population distribution map in the area of interest to self-assess the risk of infection around the area of interest.

To sum up, the method of track monitoring method and the track monitoring module provided by the present invention can be used to collect the known target tracks by the monitoring server, use the mobile device of the user end to compare whether its own track conforms to the target track, and then determine whether the mobile device of the user end is the target. In addition, the method of track monitoring method and the track monitoring module provided by the present invention can also provide a population distribution map around the mobile device of the user, thereby helping the user to evaluate the risk in a specific area.

What is claimed is:

1. A method for monitoring tracks, comprising:
maintaining a target list, stored a target track, by a monitoring server;
recording a first track related to a first mobile device by a first monitoring application; and
comparing the similarity between the first track and the target track by the first monitoring application to generate a comparison result.

2. The method for monitoring tracks according to claim 1, wherein the first mobile device is associated with a first Bluetooth code, and when the comparison result indicates that the first track matches the target track, the first monitoring application uploads the first Bluetooth code to the monitoring server.

3. The method for monitoring tracks according to claim 2, further comprising:
pushing, by the monitoring server, the first Bluetooth code to a second monitoring application associated with a second mobile device; and
determining, by the second monitoring application, whether the first Bluetooth code is recorded in a Bluetooth receiving list of the second mobile device.

4. The method for monitoring tracks according to claim 1, further comprising:
generating a reference track by the first monitoring application according to the first track when the comparison result indicates that the first track matches the target track; and
uploading the reference track, being de-identified, to the monitoring server by the first monitoring application.

5. The method for monitoring tracks according to claim 4, wherein the first track includes at least a real geographic address, the reference track includes at least a reference geographic address, and in the step of generating the reference track by the first monitoring application according to the first track, further comprising:
selecting a landmark address spaced apart by the real geographic addresses less than a first distance; and
setting the landmark address as the reference geographic address.

6. The method for monitoring tracks according to claim 5, wherein in the step of uploading the reference track, being de-identified, to the monitoring server by the first monitoring application, further comprising:
randomizing the reference geographic address to generate a plurality of second geographic addresses, each of the second geographic addresses corresponded to a weight value; and
uploading the second geographic addresses and the weight values corresponded to the second geographic addresses.

7. The method for monitoring tracks according to claim 6, further comprising:
generating, by the monitoring server, a population distribution map according to the second geographic addresses and the weight values corresponded to the second geographic addresses; and
obtaining the population distribution map by a third monitoring application associated with a third mobile device;
wherein the population distribution map records a track quantity associated with each of the landmark addresses in the target list.

8. The method for monitoring tracks according to claim 7, further comprising:
displaying, by the third monitoring application, the population distribution map within a second distance around a positioning address of the third mobile device.

9. The method for monitoring tracks according to claim 7, further comprising:
displaying, by the third monitoring application, the population distribution map of an administrative area where a positioning address of the third mobile device is located.

10. The method for monitoring tracks according to claim 1, wherein the first track includes at least a real geographic address, the target track includes at least a target geographic address, and in the step of comparing the similarity between the first track and the target track by the first monitoring application to generate the comparison result, further comprising:
determining whether the distance between the real geographic address and the target geographic address is less than a third distance; and
generating the comparison result indicating that the first track matches the target track when the distance between the real geographic address and the target geographic address is less than the third distance.

11. A track monitoring module, comprising:
a transmission unit for receiving a target track; and
a processor for executing a first monitoring application;
wherein the first monitoring application records a first track and compares the similarity between the first track and the target track to generate a comparison result.

12. The track monitoring module according to claim 11, wherein the track monitoring module is disposed in a mobile device, the mobile device is associated with a first Bluetooth code, and the first Bluetooth code is uploaded by the first monitoring application when the comparison result indicates that the first track matches the target track.

13. The track monitoring module according to claim 12, wherein the transmission unit further receives a target Bluetooth code, and the first monitoring application determines whether the target Bluetooth code is recorded in a Bluetooth receiving list of the mobile device.

14. The track monitoring module according to claim 11, wherein a reference track is generated by the first monitoring application according to the first track when the comparison result indicates that the first track matches the target track, and the first monitoring application uploads the reference track which is de-identified.

15. The track monitoring module according to claim 14, wherein the first track includes at least a real geographic address, the reference track includes at least a reference geographic address, the first monitoring application selects a landmark address spaced apart by the real geographic addresses less than a first distance, and sets the landmark address as the reference geographic address.

16. The track monitoring module according to claim 15, wherein the first monitoring application randomizes at least one of the reference geographic addresses of the reference track to generate a plurality of second geographic addresses, each of the second geographic addresses corresponded to a weight value, and the second geographic addresses and the weight values corresponded to the second geographic addresses are uploaded by the transmission unit to de-identify the reference track.

17. The track monitoring module according to claim 15, wherein the first monitoring application receives a population distribution map through the transmission unit, and the population distribution map records a track quantity associated with each of the landmark addresses in the target list.

18. The track monitoring module according to claim 17, wherein the first monitoring application displays the population distribution map within a second distance around a positioning address of the mobile device.

19. The track monitoring module according to claim 17, wherein the first monitoring application displays the population distribution map of an administrative area where a positioning address of the mobile device is located.

20. The track monitoring module according to claim 11, wherein the first track includes at least a real geographic address, the target track includes at least a target geographic address, the first monitoring application determines whether the distance between the real geographic address and the target geographic address is less than a third distance, and generates the comparison result indicating that the first track matches the target track when the distance between the real geographic address and the target geographic address is less than the third distance.
